SEQUENCE LISTING (4 sequences)

SEQ ID NO: 1 - DNA, 1918 bp, Pholiota microspora
  CDS (13)..(1890)
ccactctgagaaatgtctcgcgttgttatcactggtgtttctggcactatcgccaatcgtctcgaaatcaatgatttcgtcaagaacgacaagtttttctcgctctacattcaagctcttcaggttatgagctcagttccacctcaggaaaacgtccgatccttcttccagatcggtggtattcacggccttccttacactccttgggacggtataactggtgatcaacccttcgacccaaacactcaatggggagggtactgcactcatggatccgttctgttccctacttggcacaggccctacgttcttctctacgagcaaatcctccacaagcacgtacaggacatcgccgcgacttacaccacttccgataaggccgcttgggtgcaggctgctgctaatcttcgtcaaccgtactgggattgggctgcgaatgctgttcctcctgaccaggtcatcgtgtcgaagaaggtaacgatcaccggctccaacggacacaaagtcgaggtggacaatcctctgtaccattacaagttccatccgattgattcttcattccctcgcccatattccgagtggccgaccaccctcaggcaaccaaacagctctcggccaaatgctacggacaacgtcgcaaagttaagaaacgttcttcgtgcgtcccaggagaacataacgtctaacacgtatagcatgctcacgcgtgtacatacttggaaggcgttcagtaaccacactgttggcgatggtggaagcaccagtaacagtcttgaggccatccacgatggaattcatgttgatgttggaggaggaggacatatgggtgatcctgctgtcgctgctttcgatcctatcttcttcttgcatcactgcaacgtcgataggctcctctctctttgggcggctattaaccctggtgtctgggtcagccccggagactcagaggacgggacctttatccttccgcctgaggctcccgtcgatgtttcgactcctctgaccccgttctccaacactgagactacattctgggcttctggcggcatcacagacaccaccaaacttggctatacttaccccgagttcaacggacttgacttgggcaatgctcaagcagtcaaagcggccattggtaatatcgtcaaccgcctgtacggagcttcggttttcagtgggtttgctgctgctacgtctgctataggcgcagggtccgtggcttccctggcagcagatgttccgttggagaaggcccctgcccccgcccccgaagccgctgcccagcctcccgtccccgcccctgctcatgtggaaccagccgttcgggcagtttctgtccatgccgctgctgcccagccccatgccgagcctcctgttcatgtgtccgctggaggtcacccgtctccccatgggttctatgactggaccgcccgtatcgaattcaagaaatacgaatttgggtctagcttctctgtactcctcttcctcggccccgtcccagaggatcccgaacaatggctcgtcagtcccaacttcgttggcgcccatcacgccttcgtcaacagcgccgcagggcactgcgccaactgtcgcagtcaaggcaatgtagtggtcgaaggcttcgtccatctgactaaatacatttctgagcatgctggattgcgctcgttaaaccccgaagttgtcgagccatatctcactaatgaacttcattggagagtgttgaaggctgatggatctgtgggtcaacttgaatctcttgaagtttccgtatatggcacgcctatgaacttgcctgttggtgcgatgttccccgtccctggaaatcgtcgtcacttccacggcatcactcacgggcgcgtcggaggcagccgtcatgccattgtttaaacgttggcacgttcatcaacgtcaattt

SEQ ID NO: 2 - protein, 625 aa, Pholiota microspora
MSRVVITGVSGTIANRLEINDFVKNDKFFSLYIQALQVMSSVPPQENVRSFFQIGGIHGLPYTPWDGITGDQPFDPNTQWGGYCTHGSVLFPTWHRPYVLLYEQILHKHVQDIAATYTTSDKAAWVQAAANLRQPYWDWAANAVPPDQVIVSKKVTITGSNGHKVEVDNPLYHYKFHPIDSSFPRPYSEWPTTLRQPNSSRPNATDNVAKLRNVLRASQENITSNTYSMLTRVHTWKAFSNHTVGDGGSTSNSLEAIHDGIHVDVGGGGHMGDPAVAAFDPIFFLHHCNVDRLLSLWAAINPGVWVSPGDSEDGTFILPPEAPVDVSTPLTPFSNTETTFWASGGITDTTKLGYTYPEFNGLDLGNAQAVKAAIGNIVNRLYGASVFSGFAAATSAIGAGSVASLAADVPLEKAPAPAPEAAAQPPVPAPAHVEPAVRAVSVHAAAAQPHAEPPVHVSAGGHPSPHGFYDWTARIEFKKYEFGSSFSVLLFLGPVPEDPEQWLVSPNFVGAHHAFVNSAAGHCANCRSQGNVVVEGFVHLTKYISEHAGLRSLNPEVVEPYLTNELHWRVLKADGSVGQLESLEVSVYGTPMNLPVGAMFPVPGNRRHFHGITHGRVGGSRHAIV

SEQ ID NO: 3 - DNA, 1918 bp, Pholiota microspora
  CDS (13)..(1890)
ccactctgagaaatgtctcgcgttgttatcactggtgtttctggcactgtcgccaatcgtctcgaaatcaatgatttcgtcaagaacgacaagtttttctcgctctacattcaagctcttcaggttatgagctcagttccacctcaggaaaacgtccgatccttcttccagatcggaggtattcacggccttccttacactccttgggacggtataactggtgatcaacccttcgacccaaacactcaatggggagggtactgcactcatggatccgttctgttccctacttggcacaggccctacgttcttctctacgagcaaatcctccacaagcacgtacaggacatcgccgcgacttacaccacttccgataaggccgcttgggtgcaggctgctgctaatcttcgtcaaccgtactgggattgggctgcgaatgctgttcctcctgaccaggtcatcgcgtcgaagaaggtaacgatcaccggctccaacggacacaaagtcgaggtggacaatcctctgtaccattacaagttccatccgattgattcttcattccctcgcccatattccgagtggccgaccaccctcaggcaaccaaacagctctcggccaaatgctacggacaacgtcgcaaagttaagaaacgttcttcgtgcgtcccaggagaacataacgtctaacacgtatagcatgctcacgcgtgtacatacttggaaggcgttcagtaaccacactgttggcgatggtggaagcaccagtaacagtctcgaggccatccacgatggaattcatgttgatgttggaggaggaggacatatggctgatcctgctgtcgctgctttcgatcctatcttcttcttgcatcactgcaacgtcgataggctcctctctctttgggcggctattaaccctggtgtctgggtcagccccggagactcagaggacgggacctttatccttccgcctgaggctcccgtcgatgtttcgactcctctgaccccgttctccaacactgagactacattctgggcttctggcggcatcacagacaccaccaaacttggctatacttaccccgagttcaacggacttgacttgggcaatgctcaagcagtcaaagcggccattggtaatatcgtcaaccgcctgtacggagcttcggttttcagtgggtttgctgccgctacgtctgctataggcgcagggtccgtggcttccctggcagcagatgttccgttggagaaggcccctgcccccgcccccgaagccgctgcccagtctcccgtccccgcccctgctcatgttgaaccagccgttcgggcagtttctgtccatgccgctgctgcccagccccatgccgagcctcctgttcatgtgtccgctggaggtcacccgtctccccacgggttctatgactggaccgcccgtatcgaattcaagaaatacgaatttgggtctagcttctctgtactcctcttcctcggccccgtcccagaggatcccgaacaatggctcgtcagtcccaacttcgtgggcgcccatcacgccttcgtcaacagcgccgcagggcactgcgccaactgtcgcaatcaaggcaatgtagtggtcgaaggcttcgtccatctgactaaatacatttctgagcatgctggattgcgctcgttaaaccccgaagttgtcgagccatatcttactaatgaacttcattggagagtgttgaaggctgatggatctgtgggtcaacttgaatctcttgaagtttccgtatatggcacgcctatgaacttgcctgttggtgcgatgttccccgtccctggaaatcgtcgtcacttccacggcatcactcacgggcgcgtcggaggcagccgtcatgccattgtttaaacgttggcacgttcatcaacgtcaattt

SEQ ID NO: 4 - protein, 625 aa, Pholiota microspora
MSRVVITGVSGTVANRLEINDFVKNDKFFSLYIQALQVMSSVPPQENVRSFFQIGGIHGLPYTPWDGITGDQPFDPNTQWGGYCTHGSVLFPTWHRPYVLLYEQILHKHVQDIAATYTTSDKAAWVQAAANLRQPYWDWAANAVPPDQVIASKKVTITGSNGHKVEVDNPLYHYKFHPIDSSFPRPYSEWPTTLRQPNSSRPNATDNVAKLRNVLRASQENITSNTYSMLTRVHTWKAFSNHTVGDGGSTSNSLEAIHDGIHVDVGGGGHMADPAVAAFDPIFFLHHCNVDRLLSLWAAINPGVWVSPGDSEDGTFILPPEAPVDVSTPLTPFSNTETTFWASGGITDTTKLGYTYPEFNGLDLGNAQAVKAAIGNIVNRLYGASVFSGFAAATSAIGAGSVASLAADVPLEKAPAPAPEAAAQSPVPAPAHVEPAVRAVSVHAAAAQPHAEPPVHVSAGGHPSPHGFYDWTARIEFKKYEFGSSFSVLLFLGPVPEDPEQWLVSPNFVGAHHAFVNSAAGHCANCRNQGNVVVEGFVHLTKYISEHAGLRSLNPEVVEPYLTNELHWRVLKADGSVGQLESLEVSVYGTPMNLPVGAMFPVPGNRRHFHGITHGRVGGSRHAIV

(19) United States
(12) Patent Application Publication
(10) Pub. No.: US 2012/0219988 A1
(43) Pub. Date: Aug. 30, 2012

(54) COMPOSITION FOR POLYMERIZING A PROTEIN

(75) Inventors: Yasuko KONISHI, Ishikawa (JP); Fumio NODA, Ishikawa (JP); Ryota OHARA, Ishikawa (JP)

(73) Assignees: ISHIKAWA PREFECTURAL UNIVERSITY, Ishikawa (JP); SUGIYO CO., LTD., Ishikawa (JP)

(21) Appl. No.: 13/403,138

(22) Filed: Feb. 23, 2012

(30) Foreign Application Priority Data
Feb. 24, 2011 (JP) 2011-39048

(51) Int. Cl. C12P 21/06; C12N 9/02

(52) U.S. Cl. 435/68.1; 435/189

(57) ABSTRACT

The present invention provides a highly efficient method of polymerizing a protein and a composition of polymerizing a protein. For example, the present invention provides a method comprising contacting a tyrosinase derived from nameko mushroom (Pholiota microspora) with a protein to be polymerized. The present invention also provides a composition for polymerizing a protein, comprising a tyrosinase derived from nameko mushroom (Pholiota microspora). In the present invention, glutamyltransferase can be optionally used. In addition, the protein to be polymerized includes, but not limited to, fish meat protein, egg white protein, a soybean protein, collagen, casein and gelatin.

### TECHNICAL FIELD

The present invention relates to a field of polymerizing a protein by an enzymatic reaction. More specifically, it relates to a field of polymerizing a protein by using a tyrosinase derived from nameko mushroom (Pholiota microspora).

### BACKGROUND ART

Polymerization of a protein is largely involved in manufacture of food. It is possible to produce gel-like food or food having viscosity by polymerizing proteins such as animal flesh, fish meat, plant proteins, collagen or the like.

Representative methods used for polymerizing a protein include chemical methods such as cross-linking or modification methods such as those which utilize a reaction between an amino group comprised in a protein, and an epoxy compound, a isocyanate compound, an azide compound, an aldehyde compound, a sulfonyl chloride compound, hydroxysuccinimide or the like, together with a carboxyl compound; cross-linking or modification methods such as those which utilize a reaction between a carboxyl group comprised in a protein and a carbodiimide compound together with an amino compound; cross-linking or modification methods such as those with an alkyl halide compound, a maleimide compound, an aziridine compound or the like which utilize a reaction with a thiol group comprised in a protein; cross-linking methods with heating (G. T. Hermanson, BIOCONJUGATE TECHNIQUES, Elsevier Science (1996); Japanese Laid-Open Publication No. 11-197234; Japanese Laid-Open Publication No. 02-71749; and Japanese Laid-Open Publication No. 11-279296).

The above chemical methods have various problems such as a problem that its reaction condition remarkably differs from that in a living body, a problem that an organic solvent or special catalyst is used, or a problem that a highly active compound is used. Thus, in a medicinal use, in view of biocompatibility, an active material cannot be included at a high concentration or a washing step and the like is required after a cross-linking or modification reaction. In addition, if a highly toxic solvent, catalyst or compound is used, it is not suitable for gelation as necessary and for example, for use of gelation in a living body, it largely invades a living body. On the other hand, if a reaction condition is mild or a low active compound is used with anxiety about toxicity of a compound to be used, it requires significant time for a reaction to complete or a reaction does not sufficiently develop such that a material with property of interest sometimes cannot be obtained within a practical reaction time.

A method of polymerization without using a chemical method includes a method using an enzyme such as glutamyltransferase. However, hydrogel obtained from treatment of a known protein with glutamyltransferase is low in stability and can degrade within several days and thus has problems such as a problem that a gel is lost before it achieves its purpose as a gel.

In addition, although there is also an enzymatic polymerization method using a tyrosinase derived from mushroom in combination with glutamyltransferase (Japanese Laid-Open Publication No. 2007-23079), the tyrosinase derived from mushroom alone did not show a polymerizing activity. Even if it was used in combination with glutamyltransferase, the polymerizing activity was insufficient.

### CITATION LIST

### Patent Literature

Japanese Laid-Open Publication No. 11-197234
Japanese Laid-Open Publication No. 02-71749
Japanese Laid-Open Publication No. 11-279296
Japanese Laid-Open Publication No. 2007-23079

### Non Patent Literature

G. T. Hermanson, BIOCONJUGATE TECHNIQUES, Elsevier Science (1996)

### SUMMARY OF INVENTION

The present inventors have unexpectedly found that a tyrosinase derived from nameko mushroom (Pholiota microspora) has a very high polymerizing activity and completed the present invention.

For example, The present invention provides the followings:

### (Item 1)

A composition for polymerizing a protein, comprising a tyrosinase derived from nameko mushroom (Pholiota microspora).

### (Item 2)

The composition according to Item 1, wherein said tyrosinase derived from nameko mushroom (Pholiota microspora) is encoded by a polynucleotide selected from the group consisting of:

(a) a polynucleotide comprising a nucleotide sequence set forth in SEQ ID NO: 1 or SEQ ID NO: 3 or fragment thereof;

(b) a polynucleotide comprising a sequence encoding an amino acid sequence set forth in SEQ ID NO: 2 or SEQ ID NO: 4 or fragment thereof,

(c) a polynucleotide encoding a variant polypeptide wherein one or more amino acids have at least one mutation selected from the group consisting of substitution, addition and deletion in an amino acid sequence set forth in SEQ ID NO: 2 or SEQ ID NO: 4;

(d) a polynucleotide which hybridizes under stringent condition with the complement of the polynucleotide of any one of (a)-(c); and

(e) a polynucleotide consisting of a nucleotide sequence with at least 70%, 80%, 90%, 95%, 96%, 97%, 98% or 99% identity to the polynucleotide of any one of (a)-(c) or complement thereof;

and has a tyrosinase activity.

### (Item 3)

The composition according to Item 1, wherein said tyrosinase derived from nameko mushroom (Pholiota microspora) is encoded by a polynucleotide selected from the group consisting of:

(f) a polynucleotide consisting of a nucleotide sequence set forth in SEQ ID NO: 1 or SEQ ID NO: 3; and

(g) a polynucleotide consisting of a sequence encoding an amino acid sequence set forth in SEQ ID NO: 2 or SEQ ID NO: 4.

### (Item 4)

The composition according to Item 1, wherein said protein to be polymerized is selected from the group consisting of fish meat protein, egg white protein and a soybean protein.

### (Item 5)

The composition according to Item 1, wherein said protein to be polymerized is selected from the group consisting of collagen, casein and gelatin.

### (Item 6)

The composition according to Item 1, further comprising a glutamyltransferase.

### (Item 7)

The composition according to Item 6, wherein said glutamyltransferase is selected from the group consisting of glutamyltranspeptidase, protein-glutamine-gamma-glutamyltransferase, protein-glutamine amine gamma-glutamyltransferase, glutamotransferase and transglutaminase.

### (Item 8)

The composition according to Item 6; wherein said glutamyltransferase is transglutaminase.

### (Item 9)

A method of polymerizing a protein, comprising:

(A) contacting a tyrosinase derived from nameko mushroom (Pholiota microspora) with the protein.

### (Item 10)

The method according to Item 9, wherein said tyrosinase derived from nameko mushroom (Pholiota microspora) is encoded by a polynucleotide selected from the group consisting of:

(a) a polynucleotide comprising a nucleotide sequence set forth in SEQ ID NO: 1 or SEQ ID NO: 3 or fragment thereof;

(b) a polynucleotide comprising a sequence encoding an amino acid sequence set forth in SEQ ID NO: 2 or SEQ ID NO: 4 or fragment thereof,

(c) a polynucleotide encoding a variant polypeptide wherein one or more amino acids have at least one mutation selected from the group consisting of substitution, addition and deletion in an amino acid sequence set forth in SEQ ID NO: 2 or SEQ ID NO: 4;

(d) a polynucleotide which hybridizes under stringent condition with the complement of the polynucleotide of any one of (a)-(c); and

(e) a polynucleotide consisting of a nucleotide sequence with at least 70%, 80%, 90%, 95%, 96%, 97%, 98% or 99% identity to the polynucleotide of any one of (a)-(c) or complement thereof;

and has a tyrosinase activity.

### (Item 11)

The method according to Item 9, wherein said tyrosinase derived from nameko mushroom (Pholiota microspora) is encoded by a polynucleotide selected from the group consisting of:

(f) a polynucleotide consisting of a nucleotide sequence set forth in SEQ ID NO: 1 or SEQ ID NO: 3; and

(g) a polynucleotide consisting of a sequence encoding an amino acid sequence set forth in SEQ ID NO: 2 or SEQ ID NO: 4.

### (Item 12)

The method according to Item 9, wherein said protein is selected from the group consisting of fish meat protein, egg white protein and a soybean protein.

### (Item 13)

The method according to Item 9, wherein said protein is selected from the group consisting of collagen, casein and gelatin.

### (Item 14)

The method according to Item 9, further comprising:

(B) contacting the protein with a glutamyltransferase.

### (Item 15)

The method according to Item 14, wherein said glutamyltransferase is selected from the group consisting of glutamyltranspeptidase, protein-glutamine-gamma-glutamyltransferase, protein-glutamine amine gamma-glutamyltransferase, glutamotransferase and transglutaminase.

### (Item 16).

The method according to Item 14, wherein said glutamyltransferase is transglutaminase.

For example, the present invention provides a composition for polymerizing a protein comprising a tyrosinase derived from nameko mushroom (Pholiota microspora) which enables the tyrosinase alone to polymerize a protein. The present invention also provides a method of polymerizing a protein comprising a tyrosinase derived from nameko mushroom (Pholiota microspora) which enables the tyrosinase alone to polymerize a protein.

### BRIEF DESCRIPTION OF THE DRAWINGS

FIG. 1 is a result of SDS-PAGE in Example 3.

### EXPLANATION OF SEQUENCE LISTING

SEQ ID NO: 1: A nucleic acid sequence encoding a tyrosinase tyr1 derived from nameko mushroom (Pholiota microspora)

SEQ ID NO: 2: An amino acid sequence of a tyrosinase tyr1 derived from nameko mushroom (Pholiota microspora)

SEQ ID NO: 3: A nucleic acid sequence of a gene encoding a tyrosinase tyr2 derived from nameko mushroom (Pholiota microspora)

SEQ ID NO: 4: An amino acid sequence of a tyrosinase tyr2 derived from nameko mushroom (Pholiota microspora)

### DESCRIPTION OF EMBODIMENTS

Throughout the present specification, unless otherwise stated, a singular expression is to be understood to encompass its plural concept as well. It is also to be understood that, unless otherwise stated, the term used herein is used in the meaning usually used in the art. Thus, unless otherwise defined, all technical terms and scientific terms used herein have the same meanings as those that are generally understood by those skilled in the art which the present invention belongs. If they contradicts, the present specification (including the definition) controls.

Although the description of preferred embodiments is described below, these embodiments are exemplary of the present invention and it should be understood that the scope of the present invention is not limited to such preferred embodiments. It should be understood that those skilled in the art can also readily carry out modification, variation and the like within the scope of the present invention in reference to the preferred examples below.

As used herein, the term “tyrosinase” is an enzyme that oxidizes tyrosine to produce melanin. A method of measuring a tyrosinase activity is not particularly limited and a normal method thereof can be used for measurement.

“Nameko mushroom (Pholiota microspora)” used herein as a source of a “tyrosinase” includes, but not limited to, Pholiota microspora (Fungi, Basidiomycota, Hymenomycetes, Agaricales, Strophariaceae, Pholiota, nameko mushroom). As used herein, “a tyrosinase derived from nameko mushroom (Pholiota microspora)” refers to a tyrosinase isolated from “nameko mushroom (Pholiota microspora)” or a tyrosinase encoded by the genome of nameko mushroom (Pholiota microspora). “A tyrosinase derived from nameko mushroom (Pholiota microspora)” refers to a polypeptide encoded by a nucleotide sequence set forth in SEQ ID NO: 1 or a variant thereof, or a polypeptide comprising an amino acid sequence set forth in SEQ ID NO: 2 or a variant thereof, and includes, but not limited to, a polypeptide, for examples, which is encoded by a polynucleotide selected from the group consisting of:

(a) a polynucleotide comprising a nucleotide sequence set forth in SEQ ID NO: 1 or fragment thereof;

(b) a polynucleotide comprising a sequence encoding an amino acid sequence set forth in SEQ ID NO: 2 or fragment thereof,

(c) a polynucleotide encoding a variant polypeptide wherein one or more amino acids have at least one mutation selected from the group consisting of substitution, addition and deletion in an amino acid sequence set forth in SEQ ID NO: 2;

(d) a polynucleotide which hybridizes under stringent condition with the complement of the polynucleotide of any one of (a)-(c); and

(e) a polynucleotide consisting of a nucleotide sequence with at least 70%, 80%, 90%, 95%, 96%, 97%, 98%, or 99% identity to the polynucleotide of any one of (a)-(c) or complement thereof;

and which has a tyrosinase activity.

“Homology” of genes (for example, nucleic acid sequences, amino acid sequences or the like) herein refers to the degree of identity of two or more gene sequences to each other. In addition, identity of sequences (nucleic acid sequences, amino acid sequences or the like) herein refers to the degree of identical sequence (individual nucleic acid, amino acid or the like) in two or more comparable sequences to each other. Thus, the higher the homology of two certain genes is, the higher the identity or similarity of their sequences is. It can be examined by direct comparison of sequences or, in a case of nucleic acid, a hybridization method under stringent condition whether two kinds of genes have homology. When two gene sequences are directly compared, these genes have homology if the DNA sequences between these gene sequences are typically at least 50% identical, preferably at least 70% identical, more preferably at least 80%, 90%, 95%, 96%, 97%, 98% or 99% identical. “Similarity” of genes (for example, nucleic acid sequences, amino acid sequences or the like) herein refers to the degree of identity of two or more gene sequences to each other when a conservative substitution is deemed as positive (identical) in the homology above. Thus, if a conservative substitution exists, homology and similarity differ depending on the existence of the conservative substitution. In addition, if no conservative substitution exists, homology and similarity show the same numerical value.

In the present specification, comparison of similarity, identity and homology of amino acid sequences or nucleotide sequences are calculated with a sequence analysis tool FASTA with default parameters.

A “fragment” herein refers to a polypeptide or a polynucleotide having a sequence of length up to 1 to n−1 with respect to a full length polypeptide or polynucleotide (length n). The length of a fragment can be suitably altered depending on its purpose, and the lower limit of the length, in a case of a polypeptide, includes, for example, 3, 4, 5, 6, 7, 8, 9, 10, 15, 20, 25, 30, 40, 50 and more amino acids, and a length set forth by an integer which is not specifically listed above (for example, 11 and the like) can be also suitable as a lower limit. Furthermore, in a case of a polynucleotide, the lower limit of the length includes 5, 6, 7, 8, 9, 10, 15, 20, 25, 30, 40, 50, 75, 100 and more nucleotides, and a length set forth by an integer which is not specifically listed above (for example, 11 and the like) can be also suitable as a lower limit. Although the length of a polypeptide and a polynucleotide herein can be respectively set forth by the number of amino acids or nucleic acids as shown above, the above numbers are not absolute and it is intended that the above numbers as an upper limit or lower limit also encompass several more or several less (or, 10% more or 10% less, for example) numbers than the exact numbers as long as the same function is maintained. In order to describe such intention, the number herein can be described with “about” before it. However, it should be understood that the presence or absence of “about” herein does not influence the interpretation of a numerical value. The length of a fragment useful herein can be determined by whether at least one of the functions of a full length protein which is a standard of the fragment is retained or not.

An “isolated” biological agent (for example, nucleic acid, protein or the like) herein refers to one substantially separated or purified from other biological agents (for example, in a case that it is a nucleic acid, an agent other than a nucleic acid, and a nucleic acid comprising a nucleic acid sequence other than the nucleic acid of interest; in a case that it is a protein, an agent other than a protein, and a protein comprising an amino acid sequence other than the protein of interest; or the like) in a cell of an organism in which the biological agent is naturally present. An “isolated” nucleic acid or protein encompasses a nucleic acid or protein which is purified by a standard purification method. Thus, an isolated nucleic acid or protein encompasses a chemically synthesized nucleic acid or protein.

A “purified” biological agent (for example, a nucleic acid, protein or the like) herein refers to one wherein at least part of agents naturally accompanied with the biological agent is removed. Thus, usually, the purity of the biological agent in a purified biological agent is higher (i.e., concentrated) than that of the biological agent being normally present.

The terms “purified” and “isolated” used herein mean that preferably at least 75% by weight, more preferably at least 85% by weight, still more preferably at least 95% by weight, and most preferably at least 98% by weight of same type of biological agent exists.

A “polynucleotide which hybridizes under stringent condition” herein refers to a polynucleotide which is obtained under well-known conditions that are routinely used in the art. Such a polynucleotide can be obtained by using colony hybridization method, plaque hybridization method or Southern blot hybridization method or the like with a polynucleotide, selected from the polynucleotides of the present invention as a probe. Specifically, it means a polynucleotide which can be identified by carrying out hybridization at 65° C. in the presence of 0.7-1.0M NaCl with a filter on which DNA derived from colonies or plaques are fixed, then washing the filter under 65° C. condition with 0.1-2× concentration of SSC (saline-sodium citrate) solution (the composition of 1× concentration of SSC solution is 150 mM sodium chloride, 15 mM sodium citrate). Hybridization can be carried out in accordance with methods described in experimental textbooks such as Molecular Cloning 2nd ed., Current Protocols in Molecular Biology, Supplement 1-38, DNA Cloning 1: Core Techniques, A Practical Approach, Second Edition, Oxford University Press (1995) or the like. Here, preferably, a sequence comprising A sequence only or T sequence only is excluded from sequences which hybridize under stringent condition. A “polynucleotide which can hybridize” refer to a polynucleotide which can hybridize with another polypeptide under the hybridizing condition. A polynucleotide which can hybridize can specifically include a polynucleotide having at least 60% or more homology, preferably a polynucleotide having 80% or more homology, and more preferably a polynucleotide having 95% or more homology, with the nucleotide sequence of a DNA which encodes the polypeptide having the amino acid sequence specifically described in the present invention.

The phrase “highly stringent condition” herein refers to a condition designed to allow hybridization of DNA strands having high complementarity in their nucleic acid sequences and to exclude hybridization of DNA significantly having a mismatch. Stringency of hybridization is determined mainly by conditions such as temperature, ionic strength and denaturing agent such as formamide. An example of such “highly stringent condition” for hybridization and washing is 0.0015 M sodium chloride,
0.0015 M sodium citrate, 65-68° C. (preferably 65° C.); or 0.015 M sodium chloride, 0.0015 M sodium citrate, and 50% formamide, 42° C. For such highly stringent condition, see, Sambrook et al., Molecular Cloning: A Laboratory Manual, 2nd Edition, Cold Spring Harbor Laboratory (Cold Spring Harbor, N.Y., 1989); and Anderson et al., Nucleic Acid Hybridization: a Practical Approach, IV, IRL Press Limited (Oxford, England). If necessary, more stringent condition (for example, higher temperature, lower ionic strength, higher formamide concentration, or other denaturing agents) can be used. For the purpose of reducing nonspecific hybridization and/or background hybridization, other agents can be included in a hybridization buffer and washing buffer. Examples of such other agents include 0.1% bovine serum albumin, 0.1% polyvinyl pyrrolidone, 0.1% sodium pyrophosphate, 0.1% sodium dodecyl sulfate (NaDodSO₄ or SDS), Ficoll, Denhardt's solution, sonicated salmon sperm DNA (or other noncomplementary DNA) and dextran sulfate, but other suitable agents can be also used. The concentration and type of these additive agents can be altered without substantially affecting the stringency of hybridization condition. A hybridization experiment is usually carried out at pH 6.8-7.4 (preferably pH 7.0); but in representative ionic strength condition, the rate of hybridization is almost pH independent. See, Anderson et al., Nucleic Acid Hybridization: a Practical Approach, Chapter 4, IRL Press Limited (Oxford, England).

A factor that influences the stability of DNA duplex includes base composition, length and degree of base pair mismatch. A hybridization condition can be adjusted by those skilled in the art, by applying these variables that allow DNA with different sequence relationships to form a hybrid. The melting temperature of perfectly matched DNA duplex can be approximated with the following formula:

Tₘ(° C.)=81.5+16.6(log [Na⁺])+0.41(% G+C)−600/N−0.72(% formamide)

wherein N is length of duplex formed, [Na⁺] is molarity of sodium ion in a hybridization solution or washing solution, % G+C is percentage of (guanine+cytosine) bases in the hybrid. For incompletely matched hybrid, melting temperature decreases by about 1° C. per 1% mismatch.

The percentage of “identity”, “homology” and “similarity” of sequences (amino acid, nucleic acid or the like) herein is sought by comparing two optimally aligned sequences over comparison window. Here, an addition or deletion (that is, a gap) can be sometimes included in the portion of a polynucleotide sequence or polypeptide sequence within a comparison window when compared to a reference sequence for optimal alignment of two sequences (if the other sequence includes additions, a gap may occur, but the reference sequence here is deemed to include no addition or deletion). Identity percent is calculated by determining the number of matched positions and by determining the number of positions where the same nucleic acid bases or amino acid residues are recognized in both sequences; dividing the number of matched positions with total numbers of positions within a comparison window; and multiplying the result by 100. In the case of use in the search, the homology is evaluated using appropriate ones among those various sequence comparing algorithms and programs that are well-known in prior art. Such algorithms and programs include, but not limited to, TBLASTN, BLASTP, FASTA, TFASTA and CLUSTALW (Pearson and Lipman, 1988, Proc. Natl. Acad. Sci. USA 85(8): 2444-2448; Altschul et al., 1990, J. Mol. Biol. 215(3): 403-410; Thompson et al., 1994, Nucleic Acids Res. 22(2): 4673-4680; Higgins et al., 1996, Methods Enzymol. 266: 383-402; Altschul et al., 1990, J. Mol. Biol. 215(3): 403-410; Altschul et al., 1993, Nature Genetics 3: 266-272). In particularly preferred embodiment, homology of protein and nucleic acid sequences is evaluated with Basic Local Alignment Search Tool (BLAST) which was well-known in prior art (see, for example, Karlin and Altschul, 1990, Proc. Natl. Acad. Sci. USA 87: 2267-2268; Altschul et al., 1990, J. Mol. Biol. 215: 403-410; Altschul et al., 1993, Nature Genetics 3: 266-272; Altschul et al., 1997, Nuc. Acids Res. 25: 3389-3402). Particularly, comparison or search may be achieved by performing the following operations using five customized BLAST programs.

(1) Compare an amino acid query sequence against a protein sequence database using BLASTP and BLAST3;

(2) Compare a nucleotide query sequence against a nucleotide sequence database using BLASTN;

(3) Compare a conceptual translation product obtained by converting a nucleotide query sequence (both strands) in six reading frames, against a protein sequence database using BLASTX;

(4) Compare a protein query sequence against a nucleotide sequence database converted in all six reading frames (both strands), using TBLASTN; and

(5) Compare six-reading frame conversion product of a nucleotide query sequence, against a nucleotide sequence database converted in six reading frames, using TBLASTX.

BLAST programs are designed to identify homologous sequences by specifying similar segments called “high-score segment pairs” between an amino acid query sequence or a nucleic acid query sequence, and preferably a subject sequence obtained from a protein sequence database or a nucleic acid sequence database. It is preferable if many high-score segment pairs are identified (that is, aligned) by a scoring matrix that is well-known in prior art. Preferably, BLOSUM62 matrix (Gonnet et al., 1992, Science 256: 1443-1445, Henikoff and Henikoff, 1993, Proteins 17: 49-61) is used as the scoring matrix. This matrix is second to none as a preferable matrix, but PAM matrix or PAM250 matrix may also be used (see, for example, Schwartz and Dayhoff, eds., 1978, Matrices for Detecting Distance Relationships: Atlas of Protein Sequence and Structure, Washington: National Biomedical Research Foundation). The BLAST programs evaluate the statistical significance of all identified high-score segment pairs, and select a segment which satisfies the threshold level of the significance that a user uniquely sets up, preferably such as the homology rate unique to the user. It is preferable to evaluate the statistical significance of high-score segment pairs using Karlin's formula, which determines statistical significance (see, Karlin and Altschul, 1990, Proc. Natl. Acad. Sci. USA 87: 2267-2268).

(Modification of a Gene, Protein Molecule, Nucleic Acid Molecule or the Like)

In a protein molecule, an amino acid comprised in the sequence can be substituted with another amino acid without apparent decrease or disappearance of interactive binding capacity, for example, in a protein structure such as a cationic region or a binding site of a substrate molecule. It is an interaction capacity and property of a protein that defines the biological function of the protein. Thus, a certain amino acid substitution can be carried out in an amino acid sequence or in its DNA coding sequence level, which can result in a protein that retains its original property even after the substitution. Thus, various modification can be carried out in a peptide disclosed herein or corresponding DNA which encodes the peptide, without apparently losing biological utility.

In designing modification as above, hydrophobicity index of an amino acid can be considered. The importance of hydrophobicity index of an amino acid in imparting an interactive biological function to a protein is generally recognized in the art (Kyte J. and Doolittle, R. F., J. Mol. Biol. 157(1): 105-132, 1982). Hydrophobic property of an amino acid contributes to the secondary structure of a resulting protein, and then defines the interaction between the protein and other molecules (for example, enzyme, substrate, receptor, DNA, antibody, antigen or the like). Each amino acid is assigned hydrophobicity index based on its properties of hydrophobicity and charge. They are: isoleucine (+4.5); valine (+4.2); leucine (+3.8); phenylalanine (+2.8); cystein/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamic acid (−3.5); glutamine (−3.5); aspartic acid (−3.5); asparagine (−3.5); lysine (−3.9); and arginine (−4.5)).

It is well-known in the art that an amino acid can be substituted with another amino acid with a similar hydrophobicity index to produce a protein which still has a similar biological function (for example, a protein equivalent in an enzymatic activity). In such amino acid substitution, the hydrophobicity index is preferably within ±2, more preferably within ±1, and still more preferably within ±0.5. It is understood in the art that such amino acid substitution based on hydrophobicity is efficient.

In the art, hydrophilicity index can also be considered in designing modification. As described in U.S. Pat. No. 4,554,101, the following hydrophilicity indexes are assigned to amino acid residues: arginine (+3.0); lysine (+3.0); aspartic acid (+3.0±1); glutamic acid (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid can be substituted with another one which has a similar hydrophilicity index and can still give a biological equivalent. In such amino acid substitution, the hydrophilic index is preferably within ±2, more preferably within ±1, and still more preferably within ±0.5.

The term “conservative substitution” herein, in an amino acid substitution, refers to substitution wherein hydrophilicity index and/or hydrophobicity index of original amino acid and substitutive amino acid are similar as described above. Examples of conservative substitution include, but not limited to, for example, those with their hydrophilicity index or hydrophobicity index within ±2, preferably within ±1, more preferably within ±0.5. Thus, examples of conservative substitutions are well-known to those skilled in the art, and include, not limited to, for example, substitution within each of the following groups: arginine and lysine; glutamic acid and aspartic acid; serine and threonine; glutamine and asparagine; and valine, leucine, and isoleucine, and the like.

A “variant” herein refers to one wherein a part thereof is altered with respect to its original substance such as polypeptide or polynucleotide. Such a variant includes substitution variant, addition variant, deletion variant, truncated variant, allele variant and the like. Such a variant includes, but not limited to, those which comprise one or several substitution(s), addition(s) and/or deletion(s) or one or more substitution(s), addition(s) and/or deletion(s) with respect to their reference nucleic acid molecule or polypeptide. An allele refers to genetic variants which belong to the same gene locus and can be distinguished from each other. Thus, an “allele variant” refers to a variant with relationship of allele to a gene. Such an allele variant usually has a sequence identical or very high similarity to its corresponding allele and usually has almost same biological activity, but may rarely have a different biological activity. “Species homologue or homolog” refers to one which has homology (preferably 60% or more homology, more preferably 80% ore more, 85% or more, 90% or more, 95% or more homology) to a gene in an amino acid level or nucleotide level in a species. A method of obtaining such a species homolog is apparent from the description herein. “Orthologs” are also called as orthologous genes, and refer to genes wherein two genes are derived from speciation from a common ancestor. For example, referring to a hemoglobin gene family having multi-gene structures as an example, human and mouse alpha hemoglobin genes are orthologs but human alpha hemoglobin gene and beta hemoglobin gene are paralogs (genes resulted from gene duplication). Orthologs are useful for presuming molecule phylogenetic tree. Since ortholog can usually play a similar function in a different species to that in the original species, the ortholog of the present invention can be also useful in the present invention.

A “conservative (conservatively modified) variant” herein is applied to both of amino acid sequence and nucleic acid sequence. For a certain nucleic acid sequence, a conservatively modified variant refers to a nucleic acid which encodes identical or essentially identical amino acid sequence, and, in case of a nucleic acid not encoding an amino acid sequence, refers to essentially identical sequences. Due to degeneracy of genetic code, many functionally identical nucleic acids encode any given protein. For example, codons GCA, GCC, GCG, and GCU all encode an amino acid, alanine. Thus, at all positions where alanine is specified by a codon, the codon can be altered by any of the corresponding codons described, without altering the encoded polypeptide. Such variation of nucleic acid is “silent modification (mutation)” which is one species of conservatively modified mutations. All nucleic acid sequences herein encoding a polypeptide also describe all possible silent mutations of the nucleic acids. It is understood in the art that each codon in a nucleic acid (except for AUG, usually the only codon for methionine, and TGG, usually the only codon for tryptophan) can be modified to produce a functionally identical molecule. Thus, each silent mutation of nucleic acid encoding a polypeptide is implicitly encompassed in each sequence described. Preferably, such modification can be done so as to avoid substitution of cysteine which is an amino acid which largely influence higher-order structure of a polypeptide. Such method of modifying a nucleotide sequence includes cleavage with restriction enzyme or the like; treatments such as those with DNA polymerase, Klenow fragment, ligation with DNA ligase or the like; site specific base substitution method with synthetic oligonucleotide or the like (site-directed mutagenesis; Mark Zoller and Michael Smith, Methods in Enzymology, 100, 468-500 (1983)), but modification can be done by other methods usually used in the art of molecular biology.

In the present specification, in order to make a functionally equivalent polypeptide, addition, deletion or modification of an amino acid can also be carried out besides substitution of an amino acid. Substitution of an amino acid refers to substitution of the original peptide with one or more, for example, 1-10, preferably 1-5, more preferably 1-3 amino acids. Addition of an amino acid refers to addition of one or more, for example, 1-10, preferably 1-5, more preferably 1-3 amino acids to the original peptide chain. Deletion of amino acid refers to deletion of one or more, for example, 1-10, preferably 1-5, more preferably 1-3 amino acids from the original peptide. Amino acid modification includes, but not limited to, amidation, carboxylation, sulfation, halogenation, truncation, lipidation, phosphorylation, alkylation, glycosylation, phosphorylation, hydroxylation, acylation (for example, acetylation) or the like. An amino acid to be substituted or added may be a native amino acid, a non-native amino acid, or an amino acid analog. A native amino acid is preferred.

A protein to be polymerized in the present invention includes, but not limited to, a protein selected from the group consisting of fish meat protein, egg white protein and a soybean protein, or a protein selected from the group consisting of collagen, casein and gelatin.

The term “glutamyltransferase” as used herein is an enzyme which transfers a glutamyl group of a glutamyl compound to an amine compound, and also called as glutamyltranspeptidase, protein-glutamine-gamma-glutamyltransferase protein-glutamine amine gamma-glutamyltransferase, glutamotransferase or transglutaminase.

(A method of making a variant polypeptide) Deletion, substitution or addition (including fusion) of an amino acid of the polypeptide of the present invention can be carried out by site-directed mutagenesis, a well-known technique. Such deletion, substitution or addition of one or several amino acids can be prepared in accordance with methods described in Molecular Cloning, A Laboratory Manual, Second Edition, Cold Spring Harbor Laboratory Press (1989); Current Protocols in Molecular Biology, Supplement 1-38, John Wiley & Sons (1987-1997); Nucleic Acids Research, 10, 6487 (1982); Proc. Natl. Acad. Sci., USA, 79, 6409 (1982); Gene, 34, 315 (1985); Nucleic Acids Research, 13, 4431 (1985); Proc. Natl. Acad. Sci. USA, 82, 488 (1985); Proc. Natl. Acad. Sci., USA, 81, 5662 (1984); Science, 224, 1431 (1984); PCT WO 85/00817 (1985); Nature, 316, 601 (1985) or the like.

(A Method of Measuring a Tyrosinase Activity)

As a method of measuring a tyrosinase activity, well-known methods of measuring activity can be utilized. For example, it includes, but not limited to, a colorimetric method using t-b-catechol as a substrate.

(A Method of Measuring Glutamyltransferase Activity)

As a method of measuring glutamyltransferase activity, well-known method of measuring the activity can be utilized. The activity of transglutaminase can be measured, for example, by incorporating hydroxylamine which is a primary amine into a synthetic substrate Z-Gln-Gly, and quantifying the amount of the produced hydroxamic acid. Typically, the amount of enzyme which produces 1 micromol hydroxamic acid at 37° C., pH 6.0 for 1 minute is defined as 1 unit.

While the present invention is illustrated in detail with the Examples or the like below, the present invention is not limited thereto.

### EXAMPLES

### Example 1

### Purification of a Tyrosinase from Nameko Mushroom (Pholiota microspora)

Using nameko mushroom (Pholiota microspora) as a starting material, the tyrosinase was purified by the following procedure.

200 g of nameko mushroom (Pholiota microspora) was added 600 ml of 0.15 M NaCl, grinded with a mixer (grinded for 15 seconds×twice), disrupted with Polytron (7 sets of 30 seconds treatment and 30 seconds cooling), and centrifuged (10,000×g, 30 seconds) to recover the supernatant. 30% saturated ammonium sulfate was used to carry out ammonium sulfate fractionation (centrifuged with 10,000×g, 15 minutes). The supernatant was recovered and further subjected to ammonium sulfate fractionation with 60% saturated ammonium sulfate (centrifuged with 10,000×g, 15 minutes). The precipitate was recovered and dissolved in 50 mM Tris-HCl, pH7.2, dialyzed (dialysis with 50 mM Tris-HCl pH 7.2, twice), and centrifuged (8,000×g, 30 minutes), then the supernatant was recovered.

The supernatant was subjected to an anion exchange chromatography (DEAE-Tyopearl). 50 mM Tris-HCl (pH7.2) was used for washing and (50 mM Tris-HCl, pH7.2+0.5 M ammonium sulfate) was used for elution.

The eluate was subjected to dialysis (dialyzed with 50 mM Tris-HCl, pH7.2, twice) and concentrated with ultrafiltration to obtain a purified enzyme solution.

### Example 2

### A Method of Tyrosinase Activity

As described below, tyrosinase activity was measured by a colorimetric method using t-b-catechol as a substance. Specifically, it was as described below.

To 400 microliter of 50 mM Tris-HCl, pH7.2, 8 microliter of 20 mM t-b-catechol and 391 microliter of distilled water, 1 microliter of sample or blank (distilled water) was added to obtain a total 800 microliter of a reaction solution for measuring activity.

A cell holder of spectrophotometer was kept at 25° C. and measured under time-scan mode at 400 nm, and change in absorbance was recorded. The amount of t-b-catechol changed was calculated from the obtained spectrum to calculate enzyme unit.

### Example 3

### Polymerization of Casein

Casein was dissolved in 50 mM sodium phosphate buffer (pH 7.0). The tyrosinase enzyme solution purified in Example 1 was diluted in distilled water to 0.002 U/microliter to obtain a tyrosinase enzyme solution. Transglutaminase (Activa TG-K) was dissolved in distilled water to be 0.05 U/microliter.

Reaction solutions were prepared in accordance with the reaction conditions described in Table 1 below. “C” is a control and T1-T5 shows each enzyme solution.

TABLE 1

| | C | T1 | T2 | T3 | T4 | T5 |
|---|---|---|---|---|---|---|
| casein | 85.0 | 85.0 | 85.0 | 85.0 | 85.0 | 85.0 |
| tyrosinase | 0.0 | 15.0 | 10.0 | 7.5 | 5.0 | 0.0 |
| transglutaminase | 0.0 | 0.0 | 5.0 | 7.5 | 10.0 | 15.0 |
| distilled water | 15.0 | 0.0 | 0.0 | 0.0 | 0.0 | 0.0 |
| total | 100.0 | 100.0 | 100.0 | 100.0 | 100.0 | 100.0 |

Next, they were incubated in a water bath at 30° C. for 60 minutes, and after they reacted, they were quickly boiled to end the reaction.

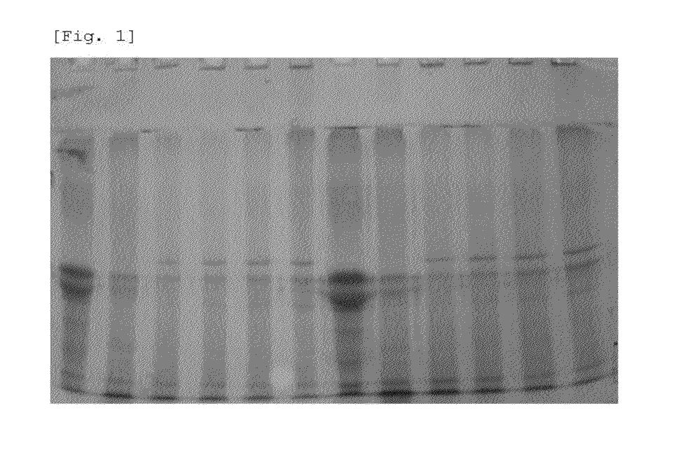

After the reaction ended, shift in casein band was confirmed by SDS-PAGE. The results are shown in FIG. 1. 1st-6th lanes from left are lanes to which 10 microliter of “C” and “T1”-“T5” were applied, respectively, and 7th-12nd lanes from left are lanes to which 20 microliter of “C” and “T1”-“T5” were applied, respectively.

The gel was measured for band intensity with GS-300™ Calibrated Densitometer (BIO-RAD) to obtain the numerical value for degree of polymerization. The results are as shown in Table 2 below.

TABLE 2

| | C | T1 | T2 | T3 | T4 | T5 |
|---|---|---|---|---|---|---|
| band intensity | 93.2 | 32.6 | 19.9 | 24.2 | 29.7 | 37.1 |
| ratio to the | — | 35.0% | 21.4% | 26.0% | 31.9% | 39.8% |
| control | | | | | | |

The intensity of the band corresponding to the position of casein was decreased compared to the control and at the same time proteins with sizes which could not invade into the gel were observed at the top of the stacking gel. Thus, it was concluded that with the T1-T5 above, casein was polymerized by the enzyme reaction. In addition, from the band intensity, the order of degree of polymerization was T2>T3>T4>T1>T5.

From the above results, even with only a tyrosinase derived from nameko mushroom (Pholiota microspora) and without using transglutaminase, a high protein polymerizing activity was observed. Such result is in contrast to the result with mushroom tyrosinase wherein no polymerizing activity. was observed with the tyrosinase only (Japanese Laid-Open Publication No. 2007-23079). It was demonstrated that the polymerizing activity of a tyrosinase derived from nameko mushroom (Pholiota microspora) was significantly higher than that of transglutaminase and thus unexpectedly superior. Furthermore, by adding transglutaminase to a tyrosinase derived from nameko mushroom (Pholiota microspora), the protein polymerizing activity was further enhanced.

### INDUSTRIAL APPLICABILITY

The present technique enables a protein which could not have been gelled to date or a protein from which only fragile gel could be made to form a strong gel and it also enables the manufacture of food with new mouthfeel.

1. A composition for polymerizing a protein, comprising a tyrosinase derived from nameko mushroom (Pholiota microspora).

2. The composition according to claim 1, wherein said tyrosinase derived from nameko mushroom (Pholiota microspora) is encoded by a polynucleotide selected from the group consisting of
(a) a polynucleotide comprising a nucleotide sequence set forth in SEQ ID NO: 1 or SEQ ID NO: 3 or fragment thereof;
(b) a polynucleotide comprising a sequence encoding an amino acid sequence set forth in SEQ ID NO: 2 or SEQ ID NO: 4 or fragment thereof,
(c) a polynucleotide encoding a variant polypeptide wherein one or more amino acids have at least one mutation selected from the group consisting of substitution, addition and deletion in an amino acid sequence set forth in SEQ ID NO: 2 or SEQ ID NO: 4;
(d) a polynucleotide which hybridizes under stringent condition with the complement of the polynucleotide of any one of (a)-(c); and
(e) a polynucleotide consisting of a nucleotide sequence with at least 70% identity to the polynucleotide of any one of (a)-(c) or complement thereof;
and has a tyrosinase activity.

3. The composition according to claim 1, wherein said tyrosinase derived from nameko mushroom (Pholiota microspora) is encoded by a polynucleotide selected from the group consisting of:
(f) a polynucleotide consisting of a nucleotide sequence set forth in SEQ ID NO: 1 or SEQ ID NO: 3; and
(g) a polynucleotide consisting of a sequence encoding an amino acid sequence set forth in SEQ ID NO: 2 or SEQ ID NO: 4.

4. The composition according to claim 1, wherein said protein to be polymerized is selected from the group consisting offish meat protein, egg white protein and a soybean protein.

5. The composition according to claim 1, wherein said protein to be polymerized is selected from the group consisting of collagen, casein and gelatin.

6. The composition according to claim 1, further comprising a glutamyltransferase.

7. The composition according to claim 6, wherein said glutamyltransferase is selected from the group consisting of glutamyltranspeptidase, protein-glutamine-gamma-glutamyltransferase, protein-glutamine amine gamma-glutamyltransferase, glutamotransferase and transglutaminase.

8. The composition according to claim 6, wherein said glutamyltransferase is transglutaminase.

9. A method of polymerizing a protein, comprising:
(A) contacting a tyrosinase derived from nameko mushroom (Pholiota microspora) with the protein.

10. The method according to claim 9, wherein said tyrosinase derived from nameko mushroom (Pholiota microspora) is encoded by a polynucleotide selected from the group consisting of
(a) a polynucleotide comprising a nucleotide sequence set forth in SEQ ID NO: 1 or SEQ ID NO: 3 or fragment thereof;
(b) a polynucleotide comprising a sequence encoding an amino acid sequence set forth in SEQ ID NO: 2 or SEQ ID NO: 4 or fragment thereof,
(c) a polynucleotide encoding a variant polypeptide wherein one or more amino acids have at least one mutation selected from the group consisting of substitution, addition and deletion in an amino acid sequence set forth in SEQ ID NO: 2 or SEQ ID NO: 4;
(d) a polynucleotide which hybridizes under stringent condition with the complement of the polynucleotide of any one of (a)-(c); and
(e) a polynucleotide consisting of a nucleotide sequence with at least 70% identity to the polynucleotide of any one of (a)-(c) or complement thereof;
and has a tyrosinase activity.

11. The method according to claim 9, wherein said tyrosinase derived from nameko mushroom (Pholiota microspora) is encoded by a polynucleotide selected from the group consisting of:
(f) a polynucleotide consisting of a nucleotide sequence set forth in SEQ ID NO: 1 or SEQ ID NO: 3; and
(g) a polynucleotide consisting of a sequence encoding an amino acid sequence set forth in SEQ ID NO: 2 or SEQ ID NO: 4.

12. The method according to claim 9, wherein said protein is selected from the group consisting of fish meat protein, egg white protein and a soybean protein.

13. The method according to claim 9, wherein said protein is selected from the group consisting of collagen, casein and gelatin.

14. The method according to claim 9, further comprising:
(B) contacting the protein with a glutamyltransferase.

15. The method according to claim 14, wherein said glutamyltransferase is selected from the group consisting of glutamyltranspeptidase, protein-glutamine-gamma-glutamyltransferase, protein-glutamine amine gamma-glutamyltransferase, glutamotransferase and transglutaminase.

16. The method according to claim 14, wherein said glutamyltransferase is transglutaminase.
